(19) United States
(12) Patent Application Publication
(10) Pub. No.: US 2009/0292306 A1
(43) Pub. Date: Nov. 26, 2009

(54) Inflatable Gastric Device and Methods Relating to the Same

(75) Inventors: Arash Kheradvar, Blythewood, SC (US); Mark H. DeLegge, Mt. Pleasant, SC (US)

Correspondence Address: DORITY & MANNING, P.A., POST OFFICE BOX 1449, GREENVILLE, SC, 29602-1449, US

(73) Assignee: University of South Carolina, Columbia, SC (US)

(21) Appl. No.: 12/472,092

(22) Filed: May 26, 2009

Related U.S. Application Data
(60) Provisional application No. 61/128,697, filed on May 23, 2008.

(51) Int. Cl. A61M 29/02

(52) U.S. Cl. 606/192

(57) ABSTRACT

In accordance with certain embodiments of the present disclosure, a device for treatment of obesity in a patient is provided. The device comprises an inflatable material and a flexible ring-shaped member. The inflatable material is joined to the flexible ring-shaped member to define a space that is configured to be expanded when the device is inflated to a generally torus shape, the ring-shaped member defining an opening therethrough.

### CROSS-REFERENCE TO RELATED APPLICATION

The present application is based on and claims priority to U.S. Provisional Application 61/128,697 having a filing date of May 23, 2008, which is incorporated by reference herein.

### BACKGROUND

Obesity is a substantial public-health crisis in the United States and in the rest of the developed world. The growing rate of obesity represents a pandemic that needs urgent attention if its potential morbidity, mortality, and economic tolls are to be avoided.

The annual cost of managing obesity in the United States alone amounts to approximately $100 billion, of which approximately $52 billion are direct costs of healthcare. These costs amount to approximately 5.7% of all health expenditures in the United States. The cost of lost productivity due to obesity is approximately $3.9 billion, and another $33 billion is spent annually on weight-loss products and services.

Although several classifications and definitions for degrees of obesity are accepted, the most widely accepted is the World Health Organization (WHO) criteria based on body mass index (BMI). Under this convention for adults, grade 1 overweight (commonly and simply called overweight) is a BMI of 25-29.9 kg/m². Grade 2 overweight (commonly called obesity) is a BMI of 30-39.9 kg/m². Grade 3 overweight (commonly called severe or morbid obesity) is a BMI greater than or equal to 40 kg/m².

The surgical literature often uses a different classification to recognize particularly severe obesity. In this setting, a BMI greater than 40 kg/m2 is described as severe obesity, a BMI of 40-50 kg/M² is termed morbid obesity, and a BMI greater than 50 kg/M² is termed super obese.

Obesity is associated with a host of potential comorbidities that significantly increase the potential morbidity and mortality associated with the condition. Amelioration of these conditions after substantial weight loss suggests that obesity probably plays an important role in their development.

Many different agents are available for the medical management of obesity. They can be classified by their mechanism of action: drugs that reduce energy intake, drugs that increase energy expenditure and nutrient-partitioning agents. Although some of these medications show promise, no long-term outcome studies have assessed their effect on overall morbidity and mortality. When the agents are discontinued, in general, weight gain follows.

Surgical therapy for obesity (bariatric surgery) is the only available therapeutic modality associated with clinically significant and sustained weight loss. The average cost of a roux-en-Y gastric bypass is $US 25,000.00. Bariatric surgery is not without its complications. Reported mortality has ranged from 1%-6%. Major complication rates of 6%-25% have been reported. Although bariatric surgery is the only therapeutic method associated with consistently demonstrable sustained weight loss it is certainly not the solution for the burgeoning obesity epidemic.

As such, a need exists for devices for the treatment of obesity. Methods relating to such devices would also be desirable.

### SUMMARY

In accordance with certain embodiments of the present disclosure, a device for treatment of obesity in a patient is provided. The device comprises an inflatable material and a flexible ring-shaped member. The inflatable material is joined to the flexible ring-shaped member to define a space that is configured to be expanded when the device is inflated to a generally torus shape, the ring-shaped member defining an opening therethrough.

In still other embodiments of the present disclosure, a method of treating obesity in a patient is provided. The method includes delivering a device into the stomach of a patient and inflating the device.

The present disclosure generally describes an expandable, intragastric device that can be used for gastrointestinal therapeutic, preventive and/or palliative purposes. The applications of the device are such as but not limited to induction of satiety or physical fullness of stomach. One aspect of usage is for treatment of obesity.

Other features and aspects of the present disclosure are discussed in greater detail below.

### BRIEF DESCRIPTION OF THE DRAWINGS

A full and enabling disclosure, including the best mode thereof, directed to one of ordinary skill in the art, is set forth more particularly in the remainder of the specification, which makes reference to the appended figures in which:

FIG. 1 illustrates a device from different perspective views in accordance with certain embodiment of the present disclosure including A) from top, B) isometric, C) from a side, D) dimetric;

FIG. 2 illustrates a human stomach with a device in accordance with the present disclosure disposed therein;

FIG. 3 illustrates a partial view of a device in accordance with certain embodiments of the present disclosure including A) a side view and B) a perspective view;

FIG. 4 illustrates a partial view of a ring-shaped member in accordance with certain embodiments of the present disclosure; and

FIG. 5 illustrates a partial view of a ring-shaped member in accordance with certain embodiments of the present disclosure including A) a side view, B) a perspective view, and C) a side view with inflatable material present.

### DETAILED DESCRIPTION

Reference now will be made in detail to various embodiments of the disclosure, one or more examples of which are set forth below. Each example is provided by way of explanation of the disclosure, not limitation of the disclosure. In fact, it will be apparent to those skilled in the art that various modifications and variations can be made in the present disclosure without departing from the scope or spirit of the disclosure. For instance, features illustrated or described as part of one embodiment, can be used on another embodiment to yield a still further embodiment. Thus, it is intended that the present disclosure covers such modifications and variations as come within the scope of the appended claims and their equivalents.

The present disclosure is generally directed to a device for treatment of obesity in a patient. The device can be inflated to a generally torus shape having an opening through the middle of the device. In this manner, the device can be delivered to the stomach while in a deflated configuration and can then be inflated in the stomach to induce satiety or physical fullness of stomach. The opening through the middle of the device not only allows alimentary fluid to pass to the pylorus but also allows an endoscope to travel inside the alimentary canal for diagnostic and/or therapeutic purposes. Further, the device is designed to allow food to pass from the stomach into the intestinal tract.

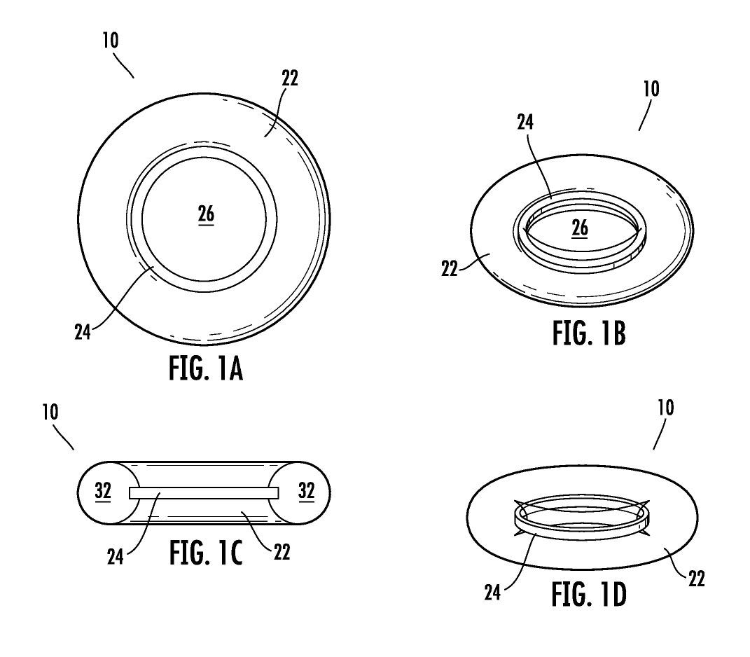

Referring to FIG. 1, a device 10 in accordance with the present disclosure is illustrated in an inflated configuration. The device includes an inflatable material 22 and a ring-shaped member 24. The inflatable material 22 is joined to the ring-shaped member 24 to define a space 32 that is expanded when the device is inflated. The inflatable material 22 can be joined to the ring-shaped member 24 by any suitable method as would be known in the art, including stitching, adhesive or the like. In certain embodiments, the inflatable material 22 and ring-shaped member 24 can be integrally molded together.

The device is designed to be inflated to a generally torus shape with the ring-shaped 24 member defining an opening 26 through the device 10. As used herein, a torus refers to a surface of revolution generated by revolving a circle in three dimensional space about an axis coplanar with the circle, which does not touch the circle. Examples of torus shapes include the surfaces of donuts and inner tubes. The present inventors have advantageously determined that such a shape permits the device of the present disclosure to be inflated in the stomach to induce satiety or physical fullness of stomach while still permitting alimentary fluid to pass to the pylorus through the opening in the middle of the device.

The inflatable material 22 can be formed from any suitable biocompatible material as would be known in the art. For example, in certain embodiments, one or more portions of the inflatable material can be formed from polyester, polypropylene, nylon, polyethylene, silicone, latex, or copolymers or combinations thereof. The material should be of sufficient size and shape to allow the device to inflate as a torus shape balloon.

Similarly, the ring-shaped member 24 can be formed from a biocompatible flexible polymer. For example, in certain embodiments, the ring-shaped member be formed from suitable polymers including polyester, polypropylene, nylon, polyethylene, silicone, latex, polyalkylene terephthalate, polyamide, polyalkene, poly(vinyl) fluoride, PTFE, polyarylethyl ketone, copolymers and combinations thereof and other such materials. In certain embodiments, the ring-shaped member can be formed of the same material(s) as the inflatable material. In addition, the ring-shaped member can be formed of a more rigid material than the inflatable material. The ring-shaped member can be of any suitable size so long as it is of sufficient size to prevent the deflated device from passing through the pylorus.

In addition to the inflatable material and the ring-shaped member, the device of the present disclosure can include one or more actuators that actuate inflation, deflation, or combinations thereof. In certain embodiments, such actuators can be configured to actuate wirelessly, by wire, or some combination thereof.

For instance, any suitable wireless protocol as would be known in the art can be utilized to actuate the actuators such as bluetooth, wireless USB, Wi-Fi, RFID, or the like. Corresponding receivers could be located on or around the actuators as would be understood in the art or could be incorporated into the actuators. In certain embodiments, the transmission of signals for controlling inflation, deflation, and combinations thereof are performed using a wireless sensor network that includes spatially distributed, autonomous, wireless communications devices implanted within the patient to transmit the data more effectively.

The actuator can be positioned on the device in any suitable location. For instance, in certain embodiments, the actuator can be located within the device in the space defined by the inflatable member and the ring-shaped member. In certain embodiments, the actuator can be positioned within the ring-shaped member. In still other embodiments, the actuator can be located on the exterior of the device so long as it does not interfere with the inflation of the device.

The actuator is configured to actuate inflation, deflation, or combinations thereof. The actuator can actuate any suitable mechanism which can control inflation, deflation, or combinations thereof in the device. In this regard, the actuator can actuate a change in temperature within the device, a change in pressure within the device, or combinations thereof that result in inflation or deflation of the device. In certain embodiments, a phase change material can be located within the device and can be configured to change from liquid to gas or from gas to liquid to inflate or deflate the device. The device described herein can be filled with a substance such as but not limited to one or more liquids with a boiling temperature close to or within the range of human body temperature. Such compounds can include, for example, certain hydrofluorocarbons. A phase change from liquid to gas under certain pressure and temperature can result in volume expansion and inflation of the torus shape device, and can eventually bring about desirable degree of inflation, with or without the use of an actuator.

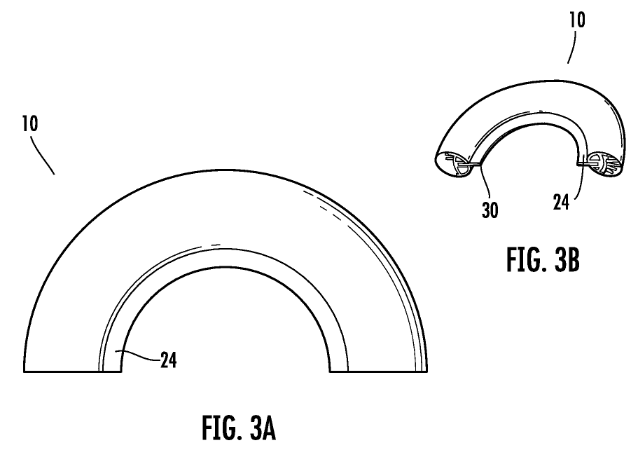

For instance, the actuator can actuate a heating element to inflate the device. Alternatively, or in combination, the actuator can actuate a condenser, a suction device, or combinations thereof to deflate the device. It should be understood that the actuator can be incorporated into the parts of the device that are being actuated. For example, referring to FIG. 3, a partial view of device 10 is shown in an inflated configuration. As shown, ring-shaped member 24 includes a heating element that includes actuator 30 and wireless circuit to inflate the device.

In certain embodiments, the device can be inflated with fluid, gas, or combinations thereof, either alone or in combination with the previously described methods. Any suitable fluid or gas can be utilized to inflate the device 10. For instance, in certain embodiments, a saline solution can be added to the device to inflate the device and expand the space defined between the inflatable material 22 and the ring-shaped member 24. A one way valve or two way valve can be incorporated into the device that enable air and fluid to be placed within the device. The valve can be connected to tubing for inflation during the delivery procedure described further herein.

Additionally, the device can be adapted to allow a patient to detect leakage or impending leakage by incorporating a dye, a pH sensitive marker, or some other biocompatible marker that will be present in the patient's excrement.

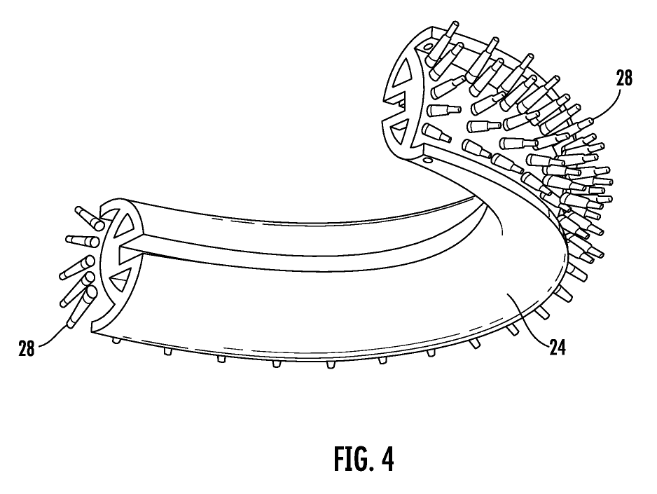

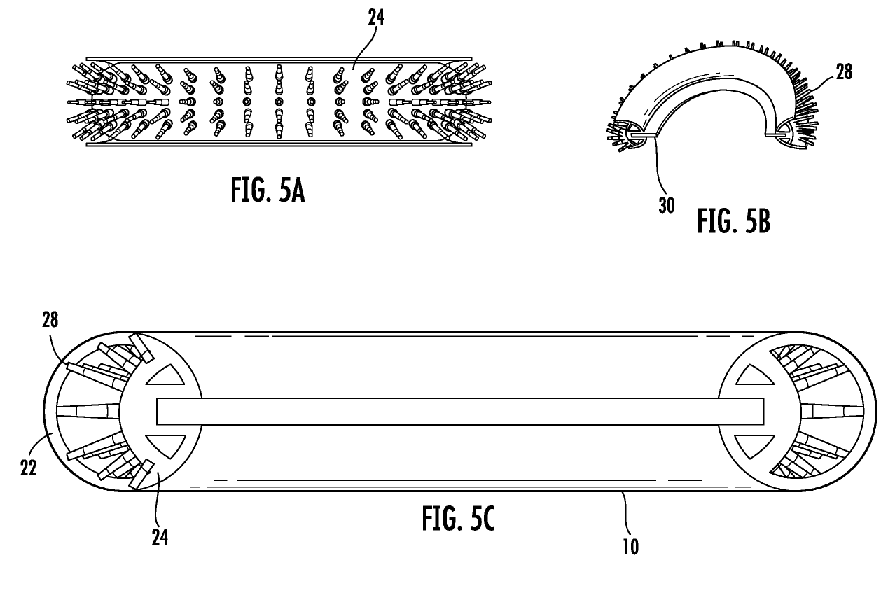

As shown in FIGS. 4-5, the device can also include one or more projections 28 that are configured to inflate the device 10. FIG. 4 illustrates the device 10 without inflatable material joined to ring-shaped member 24. Projections 28 extend from ring-shaped member 24. In certain embodiments, the projections 28 are telescoping such that they are configured to expand when inflating the device 10 and contract when deflating the device 10. The projections can either be used in connection with the fluid and/or gas and/or any of the other methods as described above or can be used alone. The projections can be formed from any suitable biocompatible material and when expanded/extended, they can push out the inflatable material as shown in FIG. 5C into the expanded torus shape. Suitable polymers can include polyester, polypropylene, nylon, polyethylene, silicone, latex, polyalkylene terephthalate, polyamide, polyalkene, poly(vinyl) fluoride, PTFE, polyarylethyl ketone, copolymers and combinations thereof and other such materials. In certain embodiments, the projections are formed from materials that have elastic and/or springy characteristics which enable them to expand or contract upon activation by an actuator.

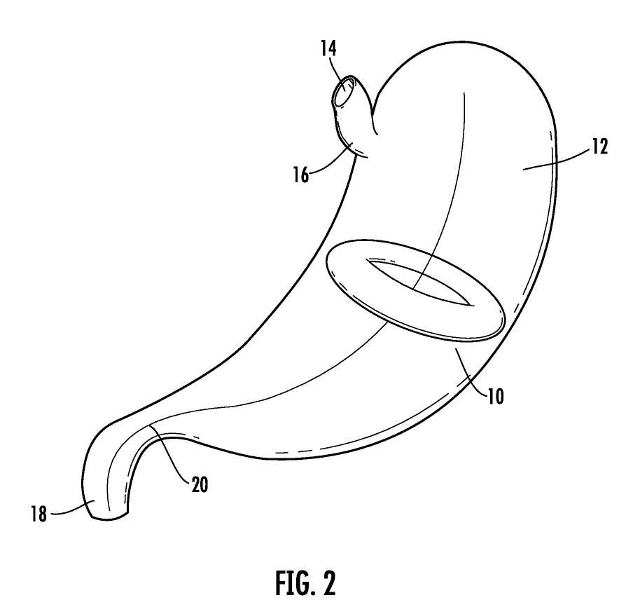

In order to better understand the operation of the device, FIG. 2 illustrates a view of a human stomach 12 with the device 10 disposed therein. The stomach 12 can be accessed via the esophagus 14 which is joined to the stomach 12 at the cardia 16. The stomach 12 is joined to the duodenum 18 (part of the small intestine) at the pylorus 20.

The size of the human stomach can be about 1.5 L or more in an adult. An adult stomach can hold from about 2 L to about 4 L of food. When a meal is consumed, the contents of the meal accumulate in the stomach. The meal is mixed with gastric juices while in the stomach and the gastric muscles contract which causes a churning movement. The stomach typically functions to temporarily store food to allow digestive enzymes to act and chemical digestion and mechanical break down to occur. The device of the present disclosure can be inflated in the stomach to induce satiety or physical fullness of stomach.

The device can be sized so as to occupy as certain percentage of a stomach when inflated. For instance, in certain embodiments, the device can occupy from about 5 percent to about 50 percent of a stomach when the device is inflated. Further, the device can be inflated to different degrees to enable further variations in the size of the device in relation to the stomach. When deflated, the device can be of sufficiently small size to fit through and throat and esophagus during delivery.

The device is deliverable to the stomach in a collapsed configuration using instruments such as but not limited to endoscope, laparoscope, or the like. For instance, the device can be placed within the stomach of a patient during an outpatient upper endoscopy procedure with conscious sedation by a gasteroenterologist. An upper endoscopy is performed and an overtube is introduced through the endoscopy into the esophagus. The endoscopy is removed and the device is inserted into the stomach via the overtube.

When the device is positioned at the desired location, it would be attached to the stomach tissue such as but not limited to muscularis mucosa, muscularis externa and/or serous membrane. In one embodiment, the device is attached to the stomach tissue with shape memory materials, including with needles such as but not limited to nitinol needles. Other materials suitable for use in connection with the device described herein include, but are not limited to biocompatible metals (such as cobalt chromium steel, surgical steels, titanium, titanium alloys, tantalum, tantalum alloys, aluminum, etc.), ceramics, polyethylene, biocompatible polymers, and other materials known in the orthopedic arts. Furthermore, surfaces may be formed from biocompatible metals such as cobalt chromium steel, surgical steel, titanium, titanium alloys (such as the nickel titanium alloy Nitinol), tantalum, tantalum alloys, aluminum, etc. Shape memory alloys, such as Nitinol, can also be used to facilitate deployment and flexibility of the device

As discussed previously, appropriate gas and/or fluid can be injected inside the torus shape device through the delivery instrument. In one embodiment, the device would be activated and its level of inflation being adjusted using the integrated control system within the device or via the delivery instrument. Further, in certain embodiments, the level of inflation can be actively controlled by the patient. In such embodiments, the patient can control the device with a wired and/or wireless control device that communicates with the implanted device. Alternatively, or in combination, such control device can be utilized to actuate the initial inflation and/or deflation if so desired. As will be appreciated, the control device can be any suitable device as would be known in the art that is capable of controlling the actuator described herein.

In the interests of brevity and conciseness, any ranges of values set forth in this specification are to be construed as written description support for claims reciting any sub-ranges having endpoints which are whole number values within the specified range in question. By way of a hypothetical illustrative example, a disclosure in this specification of a range of 1-5 shall be considered to support claims to any of the following sub-ranges: 1-4; 1-3; 1-2; 2-5; 2-4; 2-3; 3-5; 3-4; and 4-5.

These and other modifications and variations to the present disclosure can be practiced by those of ordinary skill in the art, without departing from the spirit and scope of the present disclosure, which is more particularly set forth in the appended claims. In addition, it should be understood that aspects of the various embodiments can be interchanged both in whole or in part. Furthermore, those of ordinary skill in the art will appreciate that the foregoing description is by way of example only, and is not intended to limit the disclosure.

What is claimed is:

1. A device for treatment of obesity in a patient comprising an inflatable material and a flexible ring-shaped member, the inflatable material being joined to the flexible ring-shaped member to define a space that is configured to be expanded when the device is inflated to a generally torus shape, the ring-shaped member defining an opening therethrough.

2. The device of claim 1, wherein the inflatable material, flexible ring-shaped member, or combinations thereof comprises polyester, polypropylene, nylon, polyethylene, silicone, latex, or copolymers or combinations thereof.

3. The device of claim 1, further comprising a phase change material located within the space, the phase change material configured to change from liquid to gas or from gas to liquid and to inflate or deflate the device.

4. The device of claim 1, further comprising an actuator that actuates inflation, deflation, or combinations thereof of the device.

5. The device of claim 4, wherein the actuator is configured to be controlled wirelessly.

6. The device of claim 4, wherein the actuator is configured to be controlled by wire.

7. The device of claim 4, wherein the actuator actuates a change in temperature within the space, a change in pressure within the space, or combinations thereof that results in inflation or deflation of the device.

8. The device of claim 7, wherein the actuator actuates a heating element that is positioned within the flexible ring-shaped member.

9. The device of claim 4, wherein the actuator actuates a condenser, a suction device, or combinations thereof.

10. The device of claim 1, further comprising projections, the projections extending from the ring-shaped member within the space, the projections configured to inflate or deflate the device.

11. The device of claim 10, wherein the projections are telescoping such that they are configured to expand when inflating the device and contract when deflating the device.

12. The device of claim 10, wherein the projections comprises polyester, polypropylene, nylon, polyethylene, silicone, latex, or copolymers or combinations thereof.

13. The device of claim 1, further comprising one or more shape memory anchors, such anchors configured to anchor the device to stomach tissue.

14. The device of claim 13, wherein the shape memory anchors comprise nitinol.

15. A method of treating obesity in a patient comprising:
delivering a device into the stomach of a patient, the device comprising an inflatable material and a flexible ring-shaped member, the inflatable material being joined to the flexible ring-shaped member to define a space that is configured to be expanded when the device is inflated to a generally torus shape, the ring-shaped member defining an opening therethrough;
inflating the device.

16. The method of claim 15, further comprising determining the stomach size of the patient and inflating the device to a certain size based on the stomach size of the patient.

17. The method of claim 15, further comprising deflating the device.

18. The method of claim 16, wherein delivering the device to the stomach in the patient further comprises an endoscopic procedure or a laproscopic procedure.

19. The method of claim 15, wherein alimentary fluid is capable of passing to the pylorus through the opening defined by the ring-shaped member in the device.

20. The method of claim 15, wherein the device is inflated wirelessly.
